(19) United States
(12) Patent Application Publication
(10) Pub. No.: US 2003/0022159 A1
(43) Pub. Date: Jan. 30, 2003

(54) Method for identifying intercellular protein factors

(76) Inventors: James A. Thomson, Madison, WI (US); Ren-He Xu, Madison, WI (US)

Correspondence Address: Nicholas J. Seay, Quarles & Brady LLP, 1 South Pinckney Street, P O Box 2113, Madison, WI, 53701-2113, US

(21) Appl. No.: 10/068,612

(22) Filed: Feb. 6, 2002

Related U.S. Application Data
(60) Provisional application No. 60/266,662, filed on Feb. 6, 2001.

(51) Int. Cl. C12Q 1/70; C12Q 1/68; C12N 5/08

(52) U.S. Cl. 435/5; 435/6; 435/366

(57) ABSTRACT

A method is disclosed for identifying the intercellular protein factors which cells use to provide developmental signals to adjacent cells in an organism. The method is based on the use of a phage display library of cDNA from the signaling cells. The method is being used to identify the factors produced by fibroblasts which inhibit differentiation in embryonic stem cells, but is broadly useful for identifying the proteins that signal between cells.

### CROSS-REFERENCE TO RELATED APPLICATIONS

This application claims priority from U.S. provisional patent application 60/266,662 filed Feb. 6, 2001.

### STATEMENT REGARDING FEDERALLY SPONSORED RESEARCH OR DEVELOPMENT

To be determined.

### BACKGROUND OF THE INVENTION

In higher organisms, various types of cells communicate to other cells through a series of protein factors, which are sometimes secreted by the signaling cells, are sometimes presented on the surface of the signaling cells, and are sometimes attached to the extracellular matrix. Whichever of these events occurs, the factors are recognized by the target or affected cells which then are affected in some manner, as by initiating the activation of previously inactive genes to start a cellular process. These intercellular factors are a primary means of local intercellular communication and organization.

The process of intercellular signaling is of particular interest in the developmental morphology of higher organisms. As an embryonic cell or cellular mass begins the differentiation process, various of the cells in the mass can assume one or another differentiated destiny. As the cells assume a differentiation path, they can begin to express factors appropriate to that path, and those factors are recognized by adjacent cells so that the organism can develop appropriately.

An area of active scientific inquiry is the culturing and use of human embryonic stem cells. Stem cells are undifferentiated cells capable of indefinite proliferation in culture while maintaining the ability to differentiate into many different cells types. In most current methods for maintaining stem cells in their undifferentiated state, the cells must be cultured on a so-called feeder cells layer which interacts with the stem cells through yet unknown intercellular factors to induce the stem cells to remain undifferentiated. The feeder cells are often, for example, a layer of fibroblast cells. No method was previously known for identifying the nature of the proteins expressed by the fibroblasts which mediate this effect.

### BRIEF SUMMARY OF THE INVENTION

The present invention is summarized in that a method for the identification of intercellular signaling factors is made possible through the use of a technique based on a phage display library. This technique has been verified in primate embryonic stem cells and will enable the identification of the factors presented by fibroblasts which maintain stems cells in their undifferentiated state. This technique is also applicable in general to the identification of protein signaling factors.

It is a feature of the method disclosed here that it provides a generalized method to identify intercellular signaling proteins irrespective of type of cells being investigated.

Other objects advantages and features of the present invention will be apparent from the following specification when taken in conjunction with the accompanying drawings.

### BRIEF DESCRIPTION OF THE SEVERAL VIEWS OF THE DRAWINGS

FIG. 1 is a schematic view of stem cells in culture on a fibroblast feeder layer.

FIG. 2 is a graphic representation of data from experiments described below.

FIG. 3 is a graphic representation of additional data from experiments described below.

### DETAILED DESCRIPTION OF THE INVENTION

It is revealed here that a phage display library technique can be used to identify the intercellular factors expressed by fibroblasts which act to inhibit the differentiation of stem cells in culture. The method used is based on a phage display technique which is generally applicable to the identification of intercellular protein signals. In short, the phages are used to display peptides encoded by the signaling cells, and the displaying phages are then presented to the target cells. By observing which phages bind to the target cells, the identity of signaling proteins recognized by the target cells can be discerned. This same method can be used to identify other proteins that serve as intercellular signals.

The work described here began with the effort to identify those protein factors which are secreted or presented by fibroblasts which are used as a feeder layer in the cultivation of primate embryonic stem cells. It is known that fibroblasts emit factors which permit the embryonic stem cells to remain in an undifferentiated state, but those factors are, at least as of yet, incompletely identified. The method described here was intended in part to help identify those factors, but the method is one that is generalizable to other forms of intercellular communication using protein factors. The method not dependent on whether or not the factors are secreted by the signaling cells or simply presented by them on their cell surface.

As used herein, “signaling cells” is intended to refer to the cell that creates the intercellular signaling factor. The cell that receives the factor and changes its condition based on detection of the factor is referred to either as the “target cell” or the “affected cell.” In the instance of the primate embryonic stem cell cultures, the fibroblasts feeder layer cells are the signaling cells and the embryonic stem cells themselves are the affected cells. In this instance, the signaling cells and the affected cells are in close physical proximity to each others. However, in the generalized practice of this method, the cells need not be in physical proximity to each other, as long as some mechanism for the transmission of proteins between the cells is available in their native environment.

In brief, the method of the present invention is based on a phage display technology. To use the process, mRNA is recovered from the signaling cells. The mRNA is reverse transcribed to create a cDNA library. The members of the cDNA library are then transferred into T7 phage vectors in such a way that the cDNA transferred into the phage is expressed on the exterior coat of the phage itself. The phage will display peptides expressed by the cDNA species which make up the cDNA library. The phage particles are then exposed to the target or affected cells. In those instances in which a particular phage is presenting a protein, or peptide component of a protein, which matches a receptor on the surface of the affected cells, the phage will bind to the affected or target cells. The binding may not be strong, but it is detectable. Thus by doing a wash, it is possible to separate phage which are bound to the target cells from phage which do not bind to the target cell. The binding and wash may need to be repeated more than once to achieve a desired level of purity of bound phage. More than one binding and wash steps may be needed since not all the phage may bind to the proper target in a single binding step. Then the phage which do bind to the target cells are eluted and amplified. The DNA sequences inserted in those phage which did bind can then be sequenced to identify a segment of cDNA associated with a gene or genes which is expressed by the signaling cells and detected by the target cells. The steps of washing, phage recovery and amplification are likely to be repeated three to five times in order to sufficiently segregate binding phage for analysis.

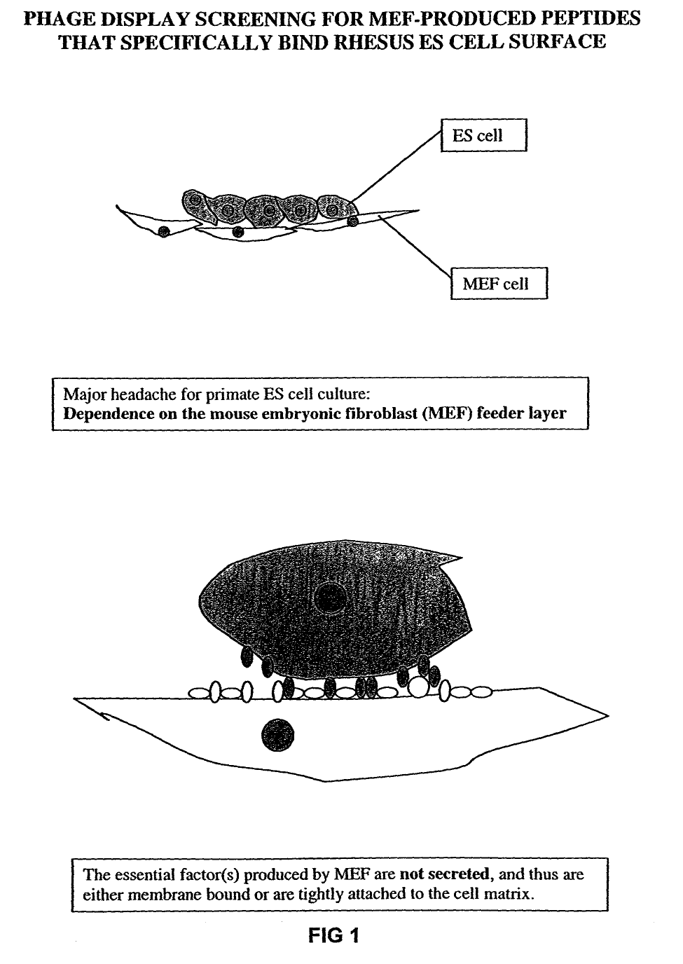

The idea behind this method is illustrated in FIG. 1. In FIG. 1, the embryonic stem cell, indicated at 10, rests on a layer of fibroblast feeder cells, indicated at 12. Protein factors, indicated at 14 and 16, pass between the stem cell 10 and the feeder cell 12. These factors signal the stem cell to remain in an undifferentiated state.

It has already become apparent that this process will yield a large number of target proteins. While it is expected that the protein signals which are responsible for the phenomenon sought to be analyzed, which in this case is the undifferentiated state of embryonic stem cells in culture, many more proteins will be identified by this assay than accomplish the objective effect. It is therefore then necessary to search among the identified proteins to find those which are sufficient for the biological effect sought to be identified.

To study the practicality of this approach, and to determine whether or not phage expressing an appropriate peptide would probably bind and could be segregated from others, a control study was done using a DSL peptide. DSL peptide is a conserved domain contained in the transmembrane ligands Delta, Serrate and Lag-2 and required for the binding to the Notch receptors, which are previously known to be present on the primate embryonic stem cells. Phage containing DSL peptides were created. Some included only the DSL region, other phages created included a DSL region plus six histidine residues or the DSL region plus an S tag (Novagen). Other phage created contained a gene for fibroblast growth factor (FGF) or contained no exogenous DNA expressing a protein whatsoever. The FGF gene was used because the primate embryonic stem cells are also known to have a receptor for that protein. The phage varieties were then exposed to primate embryonic stem cells in culture and the binding of the phage to the stem cells was quantified. The results were that essentially the DSL protein properly bound to the primate embryonic stem cells in preference over phage which did not display exogenous protein. Similar results were observed with the phage which displayed the basic fibroblast growth factor, which is consistent with the fact that human embryonic stem cells express receptors for that growth factor.

A technique known as biopanning can be used to enrich the ratio of binding phage to non-binding phage in this reaction. Essentially biopanning consists of repetitive washing, amplifying, and binding protocols so as to more clearly segregate out those phage that bind to the target cells from those that do not. Biopanning can be thought of as an enrichment process to enrich the concentration of bound phage expressing a protein signal from a given experiment. Normally three to five rounds of successive repetition of those steps would be performed. Biopanning need not be completely efficient, as long as the binding phage become more abundant rather than less abundant in each successive stage of panning. Empirical testing will reveal the number of panning steps effective to enrich for a particular binding reaction.

A fibroblast cDNA library has been created in T7 phage and four rounds of biopanning have been performed on both rhesus and human embryonic stem cells. Samples have begun to be taken of the phage plaques enriched by this biopanning process on the primate embryonic stem cells. The inserts in the phage vectors have been amplified and sequenced. Several membrane bound proteins are receptors encoded by the inserts are to be analyzed for their effects on embryonic stem cells.

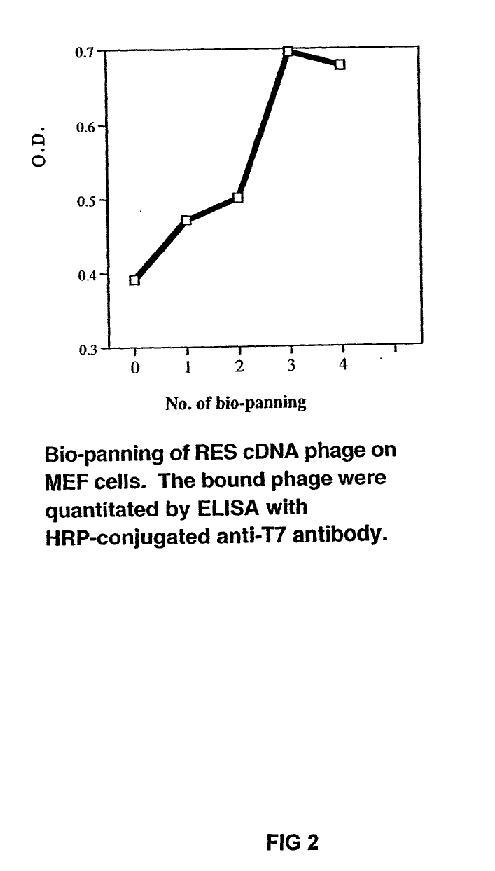

FIG. 2 illustrates in a graphical form results that demonstrate that the biopanning process enriches the proportion of phage which bind to the target molecules. Numerous genes have so far been identified, the genes being found in the sequence of DNA from phage plaques which did bind to the primate embryonic stem cells, in this case rhesus monkey embryonic stem cells. The genes identified start to create the data base of intercellular factor genes from which the identification of the actual intercellular factors responsible for any given biological effect of interest can be identified. In the instance of this experiment, the genes identified included both soluble intracellular factors, membrane bound proteins and several other non-classifiable proteins.

Note that in the examples described below, a phage library from one cell type (MEF cells) has been used to perform biopanning on the whole cell surface of another cell type (the hES cells), without prior knowledge of the ligands and the receptors from either cell type. This approach was therefore challenging because it uses both the cDNA library and the whole cell sruface to bind against. The system proved to permit the detection of some secretory and membrane bound proteins.

### EXAMPLES

Culture of hES Cells. The human embryonic stem (hES) cell line Hi was isolated and cultured by the methods generally described in U.S. Pat. No. 5,843,780. In the culture of this line, DMEM/F12 medium (GIBCO/BRL, Bethesda, Md.) was supplemented with 15% KnockOut SR serum replacer, 2 mM L-glutamine, 0.1 mM b-mercaptoethanol, and 1% nonessential amino acids. The supplemented medium was then conditioned on dense mouse embryonic fibroblast (MEF) cells overnight. Six-well plates were pre-coated with matrigel (Becton Dickinson Labware, Bedford, Mass.). H1 cells were then cultured in the conditioned medium mixed with 4 ng/ml human basic fibroblast growth hormone (bFGF) in the pro-coated plates. The cells were refreshed with the conditioned medium daily and passaged approximately weekly to maintain undifferentiated growth.

Construction of a MEF cDNA phage library and a positive control phage. PolyA mRNA was extracted from about 10⁸ non-irradiated MEF cells derived from 12 days-old mouse embryos using the Straight A's mRNA Isolation System (Novagen, Madison, WI). The purified MEF polyA mRNA was randomly primed in the first-strand synthesis followed by second-strand synthesis to yield double-stranded cDNA. The cDNA was doubly digested with EcoR I and Hind III endonucleases and size fractionated. The purified cDNA was then directionally ligated into T7Select 10-3 vector arms and subsequently in vitro packaged into phagemids (Novagen). The number of total variants of the packaged phage was 1.5×10⁷ plaque forming units (pfu) as evaluated by plaque assay according to the manufacturer's instruction (Novagen). These initial variants were subjected to one amplification step by infecting 50 ml of log-phase E. coli BLT5403 cells, providing a MEF library of cDNA phage with an overall titer of 2×10¹¹ pfu/ml. The library was stored in aliquot (500 ml) at −70° C. as 8% glycerol stocks.

To construct a positive control phage, we ligated end-modified full-length cDNA of human bFGF (gift of M. Seno) in frame into the EcoR I/Hind III arms of the T7Select 10-3 phage vector. The vector was packaged and the resultant recombinant phage (now named FGF phage) was titered, amplified, and stored as described above.

Plaque lift. Plates with around 100-300 plaques each were used for plaque lift according to the Novagen protocol. The plates were cooled down at 4° C. for more than 1 h. A round-shaped nitrocellulose membrane was placed onto each plate to fully cover the plaque for 1 min. The membranes were then carefully peeled off the plates, left to air dry for 10 min., and placed to a new plate. 10 ml of 5% milk in PBS was added to each plate to block the membrane and the plates were rocked at room temperature (RT) for 30 min. Polyclonal rabbit anti-bFGF antibodies (Santa Cruz) at 1:1,000 dilution for FGF phage or horseradish peroxidase (HRP)-conjugated S protein at 1:5,000 dilution for S-tagged phage (Novagen) were added to the blocking solution. The plates were rocked at RT for 30 min. The solution was then removed and the membranes were washed with 0.05% Tween20 in PBS (PBS-T) three times. For FGF phage, the membrane was further incubated with HRP-conjugated anti-rabbit-IgG antibodies at a dilution of 1:5,000 and rocked at RT for another 30 min., followed by 3 washes as described above. After removing the final washing solution, all the membranes were treated with ECL solutions 1 and 2 (Amersham) at 1 ml each for 5 min. The stained plaques were visualized by exposure in the Fuji Imager (Fujifilm, Inc.) for chemiluminescence emission for 5 min. The ratio of stained plaque number over total plaque number was recorded.

Biological test of peptide-displaying phage on H1 cells. 5×10¹⁰ pfu of test phage were mixed with matrigel to coat a well of 6-well plates. Control wells were coated with matrigel without phage or with the negative control phage. To each well was added 2 ml of MEF-conditioned medium containing 2 mg/ml heparin and 0.5 ml of LB medium containing 5×10¹⁰ pfu of the same phage. The conditioned medium was derived from supplemented DMEM-F12 medium conditioned by highly dense MEF overnight. H1 cell colonies were then evenly split to each of the wells. The medium was refreshed with the same medium every day. H1 cell phenotype was observed daily for 5 days and the ratio of differentiated colony number over total colony number per well was scored.

Phage preparation for biopanning. BLT5403 cells (from a 5 ml saturated culture) were added to 10 ml of LB culture and then incubated at 37° C. until cell growth reached log phase (OD₆₀₀=0.5-0.8). The culture was then inoculated with 10¹⁰ pfu of stocked phage and incubated at 37° C. until completely lysed. The lysate was centrifuged at 4500 rpm for 30 min. at 4° C. to precipitate the cell debris. The supernatant was titered before bio-panning.

Biopanning on HI cells. H1 cells were allowed to grow until about half confluence on matrigel with supplemented medium conditioned on MEF in two wells of a 6-well plate. Cells in well 1 were refreshed with the supplemented (unconditioned and also bFGF-free) DMEM/F12 medium the day before biopanning to reduce MEF-derived ligands and exogenous factors bound to the cell surface. These cells were then blocked with 5% milk in DMEM medium (GIBCO/BRL) at RT for 30 min. Cells in well 2 remained in culture with the conditioned medium plus bFGF. Phage mixture or phage library was first added to well 2 for negative selection for 1 hour and then transferred to well 1 for panning. The phage in well 1 was incubated at RT for 1 hour followed by removal of the phage. Well 1 was then washed with the supplemented medium at an increasing amount (2-5 ml) for 5 min. each time for a total of 10 times. After the final washing, 1 ml of the supplemented medium was added to the well. All or selected samples of H1 colonies in the well were scraped off with a blunt-ended Pasteur pipette under a dissecting microscope. The cell colony suspension was spun briefly to precipitate the cells followed by removal of the supernatant. 1% NP-40 100 μl was added to the cell pellet, which was followed by vortexing until total lysis was obtained. 850 μl LB medium and 50 μl Protease Inhibitor Cocktail (Sigma) were added to the lysate to achieve a total volume of 1 ml. An aliquot of the lysate was used to make serial dilutions for plaque assay in LB agar-coated plates (some of the plates with 100-300 plaques were used for plaque lift as needed). Whenever necessary, the remaining lysate was amplified and then saved for the next round of panning according to the manufacturer's instruction (Novagen). A total of 4 rounds of panning were carried out for MEF phage panning, and this was followed by sequencing analysis of the selected phage.

Sequencing of phage plaque. The phage lysate from the fourth round of panning with the MEF phage library was plated at a dilution sufficient to produce about 200 plaques per plate. Plaque pick-up, PCR amplification, and sequencing were performed according to the manufacturer's instruction (Novagen). Briefly, more than 100 plaques were randomly picked up by using a pipette tip to scrape up a portion of the top of an individual plaque and to dispense the plaque in a tube containing 100 ml of 10 mM EDTA (pH 8.0). The tube was vortexed briefly, heated to 65° C. for 10 min., cooled to RT, and centrifuged at 14,000 g for 3 min. to clarify. Inserts in the phage lysate were amplified with a pair of primers complementary to the flanking regions of the T7Select 10-3 Arms. The PCR reaction products were electrophoresized on TAE gel. Only for those PCR reactions for which the products exhibited clear single insert bands (about 200 reactions per phage lysate) were the corresponding plaques chosen for sequencing. Single chain DNAs and nucleotides left in the reactions were digested with a Pre-sequencing kit (USB, Cleveland, Ohio) followed by heat inactivation of the enzymes. An aliquot from each of the treated PCR reactions was then sequenced with a forward primer (TGC CAA TAA AGG TGA GGG TA). The sequences were visualized by the EditView software and analyzed by BLAST in the GenBank.

PCR detection of gene inserts in phage. PCR amplification of MEF cDNA inserts in the phage was performed mainly according to the protocol (Novagen). Briefly, phage DNA was purified from 2×10¹⁰ pfu of phage using a PCR purification kit (Qiagen) and an equivalence of 5×10⁶ pfu of the phage DNA was used as template per PCR reaction. PCR conditions were 3 min. at 94° C., 30 cycles (except for 18S rRNA that used 20 cycles) of 20 sec. at 94° C., 30 sec. at 55° C., and 1 min. at 72° C. Primers used were as follows: 1. Full-length inserts: forward, TGC CAA TAA AGG TGA GGG TA; and reverse, GAA TGT CGT TCA CAG CTG AT. 2. Mouse bFGF: forward, CAA AGG AGT GTG TGC TAA CCG TT; and reverse, AGA AAC ACT CAT CCG TAA CAC ATT TAG A. 3. Mouse BMP4: forward, CGT TAC CTC AAG GGA GTG GAG ATT G; and reverse, TAT TCT TCT TCC TGG ACC GCT G. 4. Mouse homolog to human PCDH19: forward, AGG CAG GGA TGG AAG GAA TAG C; and reverse, TGG ACC AGA CTT GCT GAG AGT GTC. 5. Mouse 18S rRNA: forward, CGA TAA CGA ACG AGA CTC TGG C; and reverse, CGG ACA TCT AAG GGC ATC ACA G. 6. T7 phage gene 10: forward: CAG GAC GAG ATG AGA TGG CTG TAG; and reverse, CAT TTG GGC TGG CGT AAA GTT AG. 10 μl from each PCR reaction was loaded to a TAE gel containing ethydium bromide and followed by electrophoresis. The DNA bands were visualized under UV for photography.

Construction of FGF phage. To establish and optimize a cDNA phage display system on HES cells, we first constructed a positive control phage to display a peptide that had been known to bind to H1 cells. bFGF was believed to be the best choice for construction of such a positive control phage for following reasons. First, FGF receptors are expressed on HES cells and addition of bFGF to the culture is required to maintain undifferentiated proliferation of HES cells. Secondly, the precursor of bFGF consists of 155 amino acids with 9 amino acids of secretion signal preceding the mature peptides. So bFGF is much shorter than the maximal capacity of 1,200 amino acids allowed for display on T7Select1-1 phage in low-copy (0.1-1 per phage) or T7Select10-3 phage in mid-copy number (5-15 per phage). T7Select415-1 can display as high as 415 copies of a heterologous peptide, but the peptide is limited up to 50 amino acids. We therefore chose the T7Select10-3 phage that displays mid-copy number of peptides in the present studies. The success of bFGF display on the phage was confirmed by plaque lift using polyclonal anti-bFGF antibodies. Plaques derived from bFGF phage responded positively to the antibodies, whereas plaques from wild-type phage failed to do so, in a easily visually observable result. Two dots (one blotted with bFGF protein, the other with BSA) served as positive and negative controls, respectively. Thus, the bFGF phage bound to hES cells did indeed display bFGF as expected.

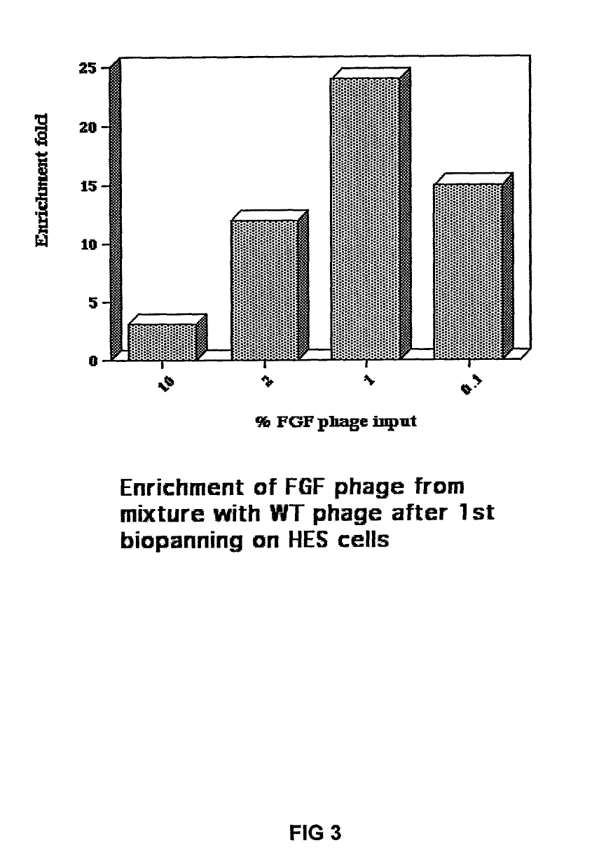

Biopanning of bFGF phage mixed with control phage. With the bFGF phage, we were able to test the panning efficiency of T7 phage display on H1 cells by using a mixture of the FGF phage with the S-tagged wild-type control phage (Novagen). Various ratios of FGF phage mixed with S tagged phage at a fixed total phage number of 10¹⁰ pfu were used to do biopanning on H1 cells. Twenty H1 colonies per well were scraped and the bound phage recovered and titered. Plaque lift was conducted on the recovered phage by using S protein to detect S tagged phage and anti-bFGF antibodies to detect bFGF phage, respectively, on two different membranes blotted from the same plaques. Enrichment of bFGF phage in the recovered phage was calculated based on the ratio of stained plaques over total plaques. FIG. 3 shows that 3.2 to 24 fold enrichment was scored for 0.1% to 10% bFGF phage input in the total phage used for biopanning, with 1% bFGF phage input yielding the highest enrichment of 24 fold. However, due a limitation on the total number of countable plaque in each plate, 0.1% input was practically the lowest ratio we could test in one round of panning. With two rounds of biopanning, FGF phage was enriched 20,000 fold from a ratio of 0.0001% (10⁻⁶) to 2%, although no enrichment was detected after 1 st round of panning.

Through these model-establishing efforts, we also optimized many other important conditions for T7 phage bio-panning on H1 cells. For example, we found that negative selection of cells in conditioned medium with bFGF prior to biopanning on cells cultured in uncoditioned medium that was bFGF-free increased bFGF phage binding to the cells. 10¹⁰ pfu of total input phage with 10% bFGF mixed with the control phage led to the highest bFGF phage recovery. Gentle rocking of the plates during biopanning was found to prevent cell detachment from the plates. Washing ten times with DMEM medium in increasing amounts (2-5 ml) for 5 min. each time reduced unbound phage to the minimum. All these optimized conditions were applied to the following biopanning with the phage library containing MEF cell cDNAs.

Biopanning of MEF phage library. Through a preliminary panning of the MEF phage library on H1 cells, we found that input phage at 10¹⁰ pfu resulted in the highest phage recovery from the cells. Since the library contained 1.5×10⁷ variants, panning with 10¹⁰ pfu of total input phage from the library would allow about 667 copies of each variant to have chance to bind H1 cells. Altogether, 4 rounds of panning were carried out, and phage recovery rate from each panning was scored. As shown in FIG. 2, the phage recovery increased 9.1-and 8.4-fold for phage after the 3 rd and 4 th rounds of panning, respectively, in comparison with the library phage. Therefore, the last two rounds of panning might represent the best yields of binding phage. It is worthwhile to notice that this enrichment is for the whole pool of binding phage, the real enrichment fold for each binding phage should vary significantly.

Sequencing of selected MEF phage. Plaque from the phage library and the 4 th round of biopanning were randomly picked up, the cDNA inserts in each plaque were PCR-amplified and sequenced. Most of the inserts ranged from 200 to 800 bp. The proportion of genes encoding secretory and membrane-bound proteins increased from 4.2% to 10.6%, and 5.6% to 25.8%, respectively from the library to the 4 th pan. Whereas, the proportion of antisenses or genes coding for intracellular proteins declined from 14.6% to 3.6%, and 39.4% to 21.2%, respectively. In light of the possibility that only part of the secretory and membrane-bound proteins involved in interaction with HES cells, the actual increasing fold for phage displaying domains of these proteins might be much higher than the average increasing fold. Among the secretory and membrane-bound proteins that had been selected were connective tissue growth factor (CTGF), Wnt16, bone morphogenetic protein 4 (BMP4), protocadherin 19 (PCDH19), fibronectin, etc.

PCR analysis of inserts in phage of each panning. Based on the sequencing information we obtained above, we decided to use PCR to test the dynamic change of the levels of some representative genes contained in phage during panning. For example, bFGF and BMP4 were chosen as representative for secreted proteins, PCDH19 for membrane-bound protein (Yoshida & Sugano, 1999), PALMA for intracellular proteins (Andreu et al., 2001), and 18S rRNA for untranslatable inserts. The encoding sequence for T7 phage gene 10 protein was tested as a loading control of the phage DNA samples in the PCR. The bFGF gene was detectable in the phage library and became clearly enriched in the 4 th panning. The BMP4 gene was also detected in the phage library and started enrichment in the 2 nd panning, while the PCDH19 gene was undetectable in the library, but gradually increased from 1 st panning. Conversely, PALMA and 18S rRNA began to decline in 2 nd and 3 rd rounds of panning, respectively, with some recovery for PALMA in the later rounds of panning. These results suggest that phage displaying peptides for secretory and membrane-bound proteins become enriched while those for intracellular proteins and untranslatable genes may become less enriched during the panning. This observation agrees with the sequencing data obtained.

Effects of the selected factors on HES cell differentiation. Despite of the clear enrichment of the growth factor-encoding sequences during panning, the final evidence for the usefulness of these factors is their biological effects on HES cells. bFGF has been known to be required for HES cells grown on MEF in serum-free medium (containing serum replacer, instead), but not required in serum-containing medium. In the absence of MEF, HES cells differentiate even in the presence of bFGF, but bFGF significantly increases the time it takes for HES cells to differentiate. Here we also observed that bFGF was required for H1 cells grown on matrigel-coated plates containing MEF-conditioned medium. In this culture, undifferentiated cells remained grainy and piled up to form comparably thick colonies. By contrast, differentiated cells turned to spread, flatten, and enlarge, thus forming colonies with the differentiated areas looking thinner and more transparent than the rest. In the absence of bFGF, the cells started to differentiate on day 4 whereas cells cultured with bFGF remained undifferentiated. Interestingly, FGF phage added to both the matrigel and the medium also prevented H1 cells from differentiation, but the negative control phage supplemented the same way failed to do so. The difference in ratios of differentiated colony number over total colony number per well became apparent between H1 cells treated with the FGF phage and those cells treated with the control phage on days 4 and 5 of the culture. FGF phage reduced the differentiation ratio from 81% to 24%. This data suggests that bFGF peptide displayed on phage, like free bFGF protein, can also suppress or delay the differentiation of H1 cells. It is worthy noting that the above selected phage that contained sequence fragments of secretory or membrane-bound proteins, or whole pools of phage from the later rounds of panning, did not alter the differentiation pace of H1 cells. This is unsurprising, though, because only some domains of the proteins displayed on phage may not be sufficient to fulfill the activity of their full-length proteins.

In addition to bFGF, another factor BMP4 is also a well-known embryonic morphogen. Previous studies have shown that BMP4 induces ectodermal and mesodermal markers in HES-derived embryonic bodies. Here we found that addition of BMP4 to the H1 cells as a monolayer induced cell differentiation with clear morphological change starting on days 2 to 3 of the treatment. Therefore, the factors we screened out from the phage display process included both agonist (BMP4) and antagonist (bFGF) of HES cell differentiation.

We claim:

1. A method for identifying proteins which function as intercellular signals between a signaling cell and affected cells, the method comprising the steps of
inserting a cDNA library from the signaling cell into a phage;
incubating the phage with the affected cells;
washing phage which does not bind to the affected cells;
eluting the phage which does bind to the affected cells; and
sequencing the cDNA inserts in the bound phage to identify sequence information useful for characterizing a protein made by the signaling cell and recognized by a receptor in the affected cells.

2. A method as claimed in claim 1 wherein the signaling cells are fibroblasts and the affected cells are embryonic stem cells.

3. A method as claimed in claim 2 wherein the embryonic stem cells are human.

4. A method as claimed in claim 1 wherein there are multiple binding and washing steps to enrich the proportion of bound phage to affected cells.

5. A method as claimed in claim 4 wherein there are two to four repetitions of the binding and washing steps.

6. A method for identifying proteins which function as intercellular signals between a fibroblast cell and embryonic stem cells cells, the method comprising the steps of
inserting a cDNA library from the fibroblast cell into phage to make a phage library;
incubating the phage library with the stem cells;
washing phage which does not bind to the stem cells;
repeating the incubating and washing steps as needed to enrich the proportion of bound phage to the stem cells;
eluting the phage which does bind to the stem cells; and
sequencing the cDNA inserts in the bound phage to derive sequence information identifying a protein made by the fibroblast and recognized by a receptor in the stem cells.
